(19) United States
(12) Patent Application Publication
(10) Pub. No.: US 2009/0202599 A1
(43) Pub. Date: Aug. 13, 2009

(54) SOLID ANTIPERSPIRANT COMPOSITION AND METHOD FOR MAKING SAME

(76) Inventors: Songtao Zhou, Mason, OH (US); David Frederick Swaile, Cincinnati, OH (US); David Arthur Sturgis, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/244,564

(22) Filed: Oct. 2, 2008

Related U.S. Application Data
(60) Provisional application No. 60/997,662, filed on Oct. 4, 2007.

(51) Int. Cl. A61K 8/06; A61K 8/19; A61Q 15/00; A61K 8/28; A61K 8/26

(52) U.S. Cl. 424/401; 424/65; 424/66; 424/68

(57) ABSTRACT

Solid emulsion antiperspirant products are described. The antiperspirant products contain a continuous phase including a water-immiscible liquid and a structurant and a disperse phase including an antiperspirant active dissolved in a polar solvent. The disperse phase has a conductivity of less than or equal to about 75 mS/cm at 25° C. And the antiperspirant active has a combined peak 4 and peak 5 area percentage of at least about 25% relative to the area sum of peaks 1 to 5. Methods for making solid emulsion antiperspirant products are also described.

### CROSS REFERENCE TO RELATED APPLICATION

This application claims the benefit of U.S. Provisional Application Ser. No. 60/997,662, filed Oct. 4, 2007.

### FIELD OF THE INVENTION

The present invention is directed to emulsified antiperspirant compositions that include a continuous phase employing a water-immiscible liquid and a structurant, and a disperse phase employing an antiperspirant active dissolved in a polar solvent. The compositions are preferably in a solid or semi-solid form. Methods for making such antiperspirant compositions are also described.

### BACKGROUND OF THE INVENTION

The wetness protection afforded by emulsion stick antiperspirant products can be limited by the efficacy of the antiperspirant active selected. Thus, it can be desirable to utilize improved efficacy antiperspirant actives known to the skilled artisan. Unfortunately, it can be difficult to manufacture emulsion stick antiperspirants with such actives since they can result in emulsion destabilization during a hot manufacturing process due to their often increased ionic strength as compared to other known actives.

### SUMMARY OF THE INVENTION

It has now been discovered that stable antiperspirant emulsions sticks can be manufactured with high efficacy antiperspirant actives through control of the conductivity level of the emulsion disperse phase.

### DETAILED DESCRIPTION OF THE INVENTION

The present invention may be understood more readily by reference to the following detailed description of illustrative and preferred embodiments. It is to be understood that the scope of the claims is not limited to the specific ingredients, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values form another embodiment. All ranges are inclusive and combinable.

All percentages and ratios used herein are by weight of the total composition, and all measurements made are at 25° C., unless otherwise designated.

The compositions/methods of the present invention can comprise, consist of, and consist essentially of the features and/or steps of the invention described herein, as well as any of the additional or optional ingredients, components, steps, or limitations described herein.

The term “ambient conditions” as used herein refers to surrounding conditions at about one atmosphere of pressure, about 50% relative humidity and about 25° C.

The term “water-immiscible” as used herein refers to materials or mixtures of materials with less than 1% water solubility at 25° C., and preferably less than 0.1% water solubility at 25° C. Most preferable are materials with less than 0.01% water solubility at 25° C.

The term “volatile” as used herein refers to those materials which have a measurable vapor pressure as measured at 25° C. and 1 atmosphere. The term “moderately volatile material,” as used herein, refers to those materials with a vapor pressure below about 2 mmHg at 25° C. The term “low volatile material,” as used herein, refers to those materials with a vapor pressure below about 0.5 mmHg at 25° C. The term “nonvolatile material,” as used herein, refers to those materials with a vapor pressure below about 0.002 mmHg at 25° C. Vapor pressures can be measured in a variety of manners and are often available in a variety of chemical data bases that would be known to one skilled in the art. One such database is available from the Research Institute for Fragrance Materials.

The term “aluminum-only active” as used herein refers to antiperspirant salts that are substantially free of zirconium.

The antiperspirant compositions of the present invention comprise a continuous phase and a disperse aqueous phase. The continuous phase includes one or more water-immiscible liquids and a structurant. The disperse phase includes an antiperspirant active dissolved in a polar solvent.

### I. Continuous Phase

A. Water-Immiscible Liquid

The concentration of the water-immiscible liquid preferably ranges from about 10% to about 30%, by weight of the composition. Other concentrations however are also contemplated herein.

One preferred water-immiscible liquid that may be employed in exemplary antiperspirant compositions that can be made in accordance with the present invention comprises volatile silicones, non-volatile silicones, or mixtures of these materials. Nonlimiting examples include those volatile silicones that are described in Todd et al., “Volatile Silicone Fluids for Cosmetics”, Cosmetics and Toiletries, 91:27-32 (1976). Suitable amongst these volatile silicones include the cyclic silicones having from about 3 or from about 4 to about 7 or to about 6, silicon atoms. Specifically are those which conform to the formula:

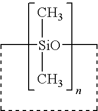

wherein n is from about 3, from about 4 or about 5 to about 7 or to about 6. These volatile cyclic silicones generally have a viscosity value of less than about 10 centistokes. Other suitable water-immiscible liquids for use herein include those volatile and nonvolatile linear silicones which conform to the formula:

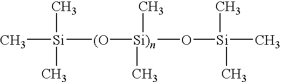

wherein n is greater than or equal to 0. The volatile linear silicone materials will generally have viscosity values of less than 5 centistokes at 25° C. The non-volatile linear silicone materials will generally have viscosity values of greater than 5 centistokes at 25° C.

Specific examples of suitable volatile silicones for use herein include, but are not limited to, hexamethyldisiloxane; Silicone Fluids SF-1202 and SF-1173 (commercially available from G.E. Silicones); Dow Corning 244, Dow Corning 245, Dow Corning 246, Dow Corning 344, and Dow Corning 345, (commercially available from Dow Corning Corp.); Silicone Fluids SWS-03314, SWS-03400, F-222, F-223, F-250, and F-251 (commercially available from SWS Silicones Corp.); Volatile Silicones 7158, 7207, 7349 (available from Union Carbide); Masil SF-V™(available from Mazer); and mixtures thereof. Examples of preferred volatile silicones include cyclohexamethylsiloxane, hexyl methicone, capryl methicone and linear or branched polydimethyl siloxanes containing 4 to 6 silicone atoms.

Specific examples of suitable non-volatile linear silicones for use herein include, but are not limited to, Rhodorsil Oils 70047 available from Rhone-Poulenc; Masil SF Fluid available from Mazer; Dow Corning 200 and Dow Corning 225 (available from Dow Corning Corp.); Silicone Fluid SF-96 (available from G.E. Silicones); Velvasil™ and Viscasil™ (available from General Electric Co.); Silicone L-45, Silicone L-530, and Silicone L-531 (available from Union Carbide); and Siloxane F-221 and Silicone Fluid SWS-101 (available from SWS Silicones).

Other suitable non-volatile silicone materials that may be employed in antiperspirant compositions manufacturable by the present invention include, but are not limited to, non-volatile silicone emollients such as polyalkylarylsiloxanes, polyestersiloxanes, polyethersiloxane copolymers, polyfluorosiloxanes, polyaminosiloxanes, and combinations thereof. These non-volatile silicone liquid carriers will generally have viscosity values of less than about 100,000 centistokes, less than about 500 centistokes, or from about 1 centistokes to about 200 centistokes or to about 50 centistokes, as measured under ambient conditions.

Silicon-free hydrophobic liquids can be employed alternatively or additionally to liquid silicones. Examples of silicon-free hydrophobic liquids include aliphatic hydrocarbons such as mineral oils, hydrogenated polyisobutane, polydecene, paraffins, isoparaffins, and aliphatic ethers derived from at least one fatty alcohol (e.g., PPG-3 myristeyl ether and PPG-14 butyl ether).

Other hydrophobic liquids include aliphatic or aromatic esters. Exemplary aliphatic esters contain at least one long chain alkyl group, such as ester derived from C1 to C20 alkanols esterified with a C8 to C22 alkanoic acid or C6 to C10 alkanedioic acid. The alkanol and acid moieties or mixtures thereof are preferably selected such that they each have a melting point of below 20° C. These esters include isopropyl myristate, lauryl myristate, isopropyl palmitate, diisopropyl sebacate and diisopropyl adipate. Exemplary aromatic esters include fatty alkyl benzoates.

Water-immiscible liquids other than those disclosed above may also be employed by the present invention. Further, it is to be understood that the continuous phase may contain hydrophilic materials, so long as the continuous phase overall is water-immiscible.

B. Structurant

Suitable structurants include polyethylene waxes, ozokerite waxes, carnuba waxes, and mixtures thereof. Other suitable structurant materials include N-acyl amino acid amides and esters; for example, N-Lauroyl-L-glutamic acid di-n-butylamide. These materials are described in greater detail in U.S. Pat. No. 3,969,087. 12-hydroxystearic acid and esters and amines of the same represent another class of useful structurants for the antiperspirant compositions of the present invention.

Fiber-forming structurants may also be employed. These materials create a network of fibers or strands that extend throughout the continuous phase to gel the liquids therein. Such materials are generally non-polymeric, being monomers or dimmers that can have a molecular weight below about 10,000. Exemplary fiber-forming structurant materials have been reviewed by Terech and Weiss in “Low Molecular Mass Gelators of Organic Liquids and the Properties of their Gels” Chem. Rev 97, 3133-3159 [1997] and by Terech in Chapter 8, “Low-molecular Weight Organogelators” of the book “Specialist Surfactants” edited by I. D. Robb, Blackie Academic Professional, 1997.

Another suitable structurant is a partially or fully esterified cellobiose according the following formula:

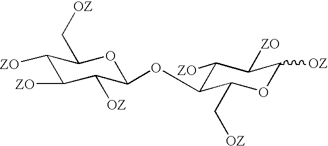

wherein each Z is independently hydrogen or an acyl group of the formula:

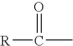

where R denotes a hydrocarbyl group containing from 4 to 22 carbon atoms. It one embodiment, not more than half of the Z groups are hydrogen.

Other suitable thickening or structuring agents for use in the present invention include, but are not limited to, fatty acid gellants, salts of fatty acids, hydroxy fatty acid gellants, esters and amides of fatty acid or hydroxy fatty acid gellants, cholesterolic materials, dibenzylidene alditols, lanolinolic materials, fatty alcohols, and triglycerides.

Suitable thickening or structuring agents can include, but are not limited to, solid salts of fatty acids wherein the fatty acid moiety has from about 12, from about 16 or from about 18 carbon atoms to about 40, to about 22, or about 20 carbon atoms. Suitable salt forming cations for use with these thickening or structuring agents include metal salts such as alkali metals (e.g. sodium and potassium), alkaline earth metals (e.g. magnesium), and aluminum. Preferred are sodium, potassium and aluminum salts. For example, suitable salt forming cations may be selected from the group consisting of sodium stearate, sodium palmitate, potassium stearate, potassium palmitate, sodium myristate, aluminum monostearate, and combinations thereof.

### II. Disperse Phase

The disperse phase generally includes water and an antiperspirant active dissolved in a polar solvent, such as, for example, water ethanol or a liquid polyol. The concentration of the antiperspirant active in the composition should be sufficient to provide the finished antiperspirant product with the desired perspiration wetness and odor control. Exemplary antiperspirant active concentrations range include from about 0.1% to about 26%, from about 1% to about 20%, and from about 2% to about 10%, by weight of the composition. All such weight percentages are calculated on an anhydrous metal salt basis exclusive of water and any complexing or buffering agent such as, for example, glycine, glycine salts.

Suitable antiperspirant actives for use in the antiperspirant compositions of the present invention may include any compound, composition or other material having antiperspirant activity. Antiperspirant actives may include astringent metallic salts, especially the inorganic and organic salts of aluminum, zirconium and zinc, as well as mixtures thereof. Particularly beneficial are believed to be salts such as aluminum halides, aluminum chlorohydrate, aluminum hydroxyhalides, zirconyl oxyhalides, zirconyl hydroxyhalides, and mixtures thereof.

Aluminum salts for use in the antiperspirant compositions may include those that conform to the formula:

Al₂(OH)ₐCl_{b}.xH₂O

wherein a is from about 2 to about 5; the sum of a and b is about 6; x is from about 1 to about 6; and wherein a, b, and x may have non-integer values. One example is the aluminum chlorohydrates referred to as “⅚ basic chlorohydrate”, wherein a=5, and “⅔ basic chlorohydrate” wherein a=4. Processes for preparing aluminum salts are disclosed in U.S. Pat. No. 3,887,692, Gilman, issued Jun. 3, 1975; U.S. Pat. No. 3,904,741, Jones et al., issued Sep. 9, 1975; and U.S. Pat. No. 4,359,456, Gosling et al., issued Nov. 16, 1982. Mixtures of aluminum salts are described in British Patent Specification 1,347,950, Shen et al., published Feb. 27, 1974.

Zirconium salts for use in the antiperspirant compositions may include those that conform to the formula:

ZrO(OH)₂₋ₐClₐ.xH₂O

wherein a is any number having a value of from 0 to about 2; x is from about 1 to about 7; and wherein a and x may both have non-integer values. Zirconium salts that additionally contain aluminum and glycine, commonly known as ZAG complexes, may also be used. These ZAG complexes contain aluminum chlorhydroxide and zirconyl hydroxy chloride conforming to the above-described formulas. Such ZAG complexes are described in U.S. Pat. No. 3,679,068, Luedders et al., issued Feb. 12, 1974; Great Britain Patent Application 2,144,992, Callaghan et al., published Mar. 20, 1985; U.S. Pat. No. 4,120,948, Shelton, issued Oct. 17, 1978 and U.S. Pat. No. 6,136,302, Juneja, issued Oct. 24, 2000.

Specific antiperspirant actives may include aluminum chlorohydrate, aluminum dichlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum sulfate buffered, aluminum zirconium trichlorohydrate, aluminum zirconium tretrachlorohydrate, aluminum zirconium pentachlorohydrate, aluminum zirconium octachlorohydrate, aluminum zirconium trichlorohydrdrex glycine, aluminum zirconium tretrachlorohydrex glycine, aluminum zirconium pentachlorohydrex glycine, aluminum zirconium octachlorohydrex glycine and combinations thereof.

One preferred species of antiperspirant actives includes aluminum only and aluminum-zirconium actives having a combined peak 4 and peak 5 area percentage of at least about 25% relative to the area sum of peaks 1 to 5, as determined by the gel permeation chromatography (“GPC”) methodology described below. Such preferred actives are disclosed in U.S. Pat. Nos. 6,245,325; 6,902,723; and 6,923,952. The antiperspirant actives employed in antiperspirant compositions herein can contain a stabilizer such as, for example, a calcium salt, a strontium salt, or mixture thereof, to maintain their efficacy during and after their manufacture.

The GPC methodology can be performed as follows: the antiperspirant active salt samples are dissolved in 0.01M nitric acid (which is also used as the mobile phase for the analysis) and chromatographed using 5 μl injections in a series of three consecutive Waters μ Porasil Columns, 3.9×300 mm, 10 μm packing. Samples should be diluted to produce an approximately 1% solution of active. A 1 mL per minute flow rate is recommended. Chromatograms are visualized using a Waters 410 Differential Refractometer. Samples are prepared immediately prior to analysis to prevent degradation. Relative peak areas and area ratios are calculated using a Waters Millennium Data System (Version 2.10 or equivalent). The peaks observed in the chromatogram are designated in order of appearance on the chromatogram as peaks 1-2 (appears as a single peak) and peaks 3, 4 and 5. The area of peaks 3, 4 and 5 correspond to the relative concentration of aluminum polymer species exiting the column during the specified time period from the injected sample. For aluminum and zirconium actives the area of peaks 1-2 corresponds to the relative concentration of co-eluting aluminum and zirconium polymer species appearing initially on the chromatogram.

Prior to any analysis, the columns should be conditioned individually by repeated 100 μl injections of a 10% zirconium-aluminum trichlorohydrate glycine solution (containing at least 10% zirconium on a solid basis). Conditioning is complete when the area percent of peaks 1-2 become relatively constant. During the conditioning process, the area percent of peaks 1-2 will increase, and there will be reduction in retention for all peaks. Columns should be discarded when peaks 1 and 2 are no longer resolved from peak 3.

The salts for the present invention may exhibit a combined peak 4 and/or peak 5 level that is greater than 25% of the total area of the chromatogram and preferably more than 30%. It should be noted, and known to one skilled in the art, that for aluminum only actives (i.e. aluminum chlorohydrate) may not contain any peak 1-2 so peak identification should be made by comparison to an appropriate standard.

It is believed that antiperspirant emulsions with high ionic strengths can become unstable when heated to or above the temperature necessary to melt the added structurant. Since ionic strength is increased by employing smaller molecular species in the antiperspirant active, the use of actives with high levels of “right-side peaks” (e.g., peaks 4 and 5), may exacerbate the emulsion stability issue. It has now been discovered that by controlling the conductivity (as an indicator of ionic strength) of the disperse phase, it is possible to create a stable emulsion stick comprising a high efficacy active, such as those with high levels of “right-side peaks.” In accordance with this discovery, preferred embodiments of the present invention have a disperse phase which has a conductivity of less than or equal to about 75 mS/cm at 25° C., or less than or equal to about 60 mS/cm at 25° C. Emulsions having a disperse phase with conductivity levels of greater than 75 mS/cm at 25° C. may be unsuitable for commercial products because of the possibility of the emulsion breaking. Conductivity of the disperse phase may be determined prior to making an emulsion and final product, or by breaking an emulsion as found in a final product, followed by analysis of the separated disperse phase components. To break the emulsion of a finished product, one skilled in the art will recognize several options, depending on the composition, including, but not limited to, solvent extraction, freezing and, in some cases, heating. The skilled artisan will choose an appropriate method that does not change the conductivity of the emulsion disperse phase.

Conductivity can be determined with various apparatuses and methods, including a Symphony Bench Top Conductivity Meter (SB70C). Prior to taking conductivity measurements of active solution, one should calibrate the chosen apparatus. To calibrate the Symphony Bench Top Conductivity Meter, the operator should first prepare the probe according to the probe user guide or operator's manual. Next, select “Cond” for conductivity measurement, and press the calibration button. Rinse the probe and place into a first conductivity standard having relatively low ionic strength (e.g., 9.96 mS/cm). Wait for the mS/cm icon to stop flashing, it will display the measured conductivity. Use the up and down key to enter the actual value of the conductivity at the measured temperature. Press the calibration key again to proceed to the next calibration point. Rinse the probe and place into a second conductivity standard having relatively high ionic strength (e.g., 100.1 mS/cm). Wait for the mS/cm icon to stop flashing, it will display the measured conductivity. Use the up and down key to enter the actual value of the conductivity at the measured temperature. Press measurement/save key to save and end calibration. To take a conductivity measurement, rinse the probe and place to into the sample. Press the measurement key and wait for mS/cm icon to stop flashing indicating a stable value. Temperature is displayed in the left corner of the display.

Controlling conductivity (and hence ionic strength) of the disperse phase may be achieved by manipulation of antiperspirant active concentration, manipulation of active composition including metal ion type, manipulation of the degree of neutralization (metal to chloride ratio), and/or addition of ionic additives, such as, for example, propylene glycol.

The antiperspirant compositions provided herein may additionally employ a deodorant active; alternatively meaning that a deodorant active is substituted for an antiperspirant active. Suitable deodorant actives may be selected from the group consisting of antimicrobial agents (e.g. bacteriocides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

The disperse phase may optionally contain other polar materials. A representative, non-limiting list of optional polar materials includes C1 to C20 monohydric alcohols; C2 to C40 dihydric or polyhydric alcohols; alkyl ethers of all such alcohols, e.g., C1-C4 alkyl ethers; polyalkoxylated glycols, e.g., propylene glycols and polyethylene glycols having from 2 to 30 repeating alkoxylate (e.g., ethoxylate or propoxylate) groups and polyglycerols having from 2 to 16 repeating glycerol moieties; and mixtures thereof. More particular exemplary polar materials include propylene glycol, hexylene glycol, dipropylene glycol, tripropylene glycol, glycerin, propylene glycol methyl ether, dipropylene glycol methyl ether, ethanol, n-propanol, n-butanol, t-butanol, 2-methoxyethanol, 2-ethoxyethanol, ethylene glycol, isopropanol, isobutanol, 1,4-butylene glycol, 2,3-butylene glycol, trimethylene glycol, 1,3-butanediol, 1,4,-butanediol, propylene glycol monoisostearate, PPG-3 myristyl ether, PEG-4 (also known as PEG-200), PEG-8 (also known as PEG-400), 1,2, pentanediol, PPG-14 butylether, dimethyl isosorbide, 1,2 hexanediol and combinations thereof. It is to be understood that polar materials other than those listed above may also be employed in the antiperspirant compositions described herein.

### III. Surfactants

Emulsifying surfactants are employed in the antiperspirant compositions to facilitate the formation of a stable emulsion containing the above-described continuous phase and disperse phase. The emulsifying surfactants may be anionic, cationic, zwitterionic and/or nonionic surfactants. Nonionic surfactants are preferred in the current invention. The proportion of emulsifier in the composition is often selected in the range up to 10% by weight and in many instances from 0.1 or 0.25 up to 5% by weight of the composition. Most preferred is an amount from 0.1 or 0.25 up to 3% by weight. Emulsifiers are frequently classified by HLB value. It is desirable, although not required, to use an emulsifier or a mixture of emulsifiers with an overall HLB value in a range from 2 to 10 preferably from 3 to 8.

It may be convenient to use a combination of two or more emulsifiers which have different HLB values above and below the desired value. By employing the two emulsifiers together in appropriate ratio, it is readily feasible to attain a weighted average HLB value that promotes the formation of an emulsion.

Many suitable emulsifiers of high HLB are nonionic ester or ether emulsifiers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditol as hydrophilic moiety. The hydrophilic moiety can contain polyoxypropylene. The emulsifiers additionally contain a hydrophobic alkyl, alkenyl or aralkyl moiety, normally containing from about 8 to 50 carbons and particularly from 10 to 30 carbons. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil, sunflower seed oil or soya bean oil. Such nonionic surfactants can also be derived from a polyhydroxy compound such as glycerol or sorbitol or other alditols. Examples of emulsifiers include ceteareth-10 to −25, ceteth-10-25, steareth-10-25 (i.e. C16 to C18 alcohols ethoxylated with 10 to 25 ethylene oxide residues) and PEG-15-25 stearate or distearate. Other suitable examples include C10-C20 fatty acid mono, di or tri-glycerides. Further examples include C18-C22 fatty alcohol ethers of polyethylene oxides (8 to 12 EO).

Examples of emulsifiers, which typically have a low HLB value, often a value from 2 to 6 are fatty acid mono or possibly diesters of polyhydric alcohols such as glycerol, sorbitol, erythritol or trimethylolpropane. The fatty acyl moiety is often from C14 to C22 and is saturated in many instances, including cetyl, stearyl, arachidyl and behenyl. Examples include monoglycerides of palmitic or stearic acid, sorbitol mono or diesters of myristic, palmitic or stearic acid, and trimethylolpropane monoesters of stearic acid.

A particularly desirable class of emulsifiers comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers also include C1 to C12 alkyl groups as functional groups. Examples of suitable surfactants include DC5225 and DC 5200 (from Dow Corning), Abil EM 90 and EM 97 (from Gold Schmidt) and KF 6026, KF 6028, KF 6038 (from Shinetsu Silicones).

The skilled artisan should appreciate that other emulsifying surfactants than those described above may also be used in antiperspirant compositions described herein.

### IV. Formation of the Emulsion

The continuous phase, disperse phase, and emulsifying surfactant are combined and then mixed or otherwise agitated sufficiently to form an emulsion. Typically, the disperse phase is added slowing to the continuous phase while the continuous phase is being vigorously agitated with a mixing system. The skilled artisan should appreciate the degree of mixing needed based on the desired phase ratio of the emulsion, its resulting viscosity and the desired batch size. The resulting emulsion can be further processed to create a consistent droplet size within the emulsion; for example, the emulsion may be processed by a mill to reduce droplet size and/or improve droplet size uniformity. Preferably, the emulsion is processed so that the entire batch experiences an equivalent amount of shear. A single-phase inline mill is one preferred apparatus for the additional, optional processing.

### V. Optional Ingredients

Antiperspirant compositions of the present invention may include one or more fragrance/perfume materials. In one preferred embodiment, the composition includes a fragrance material comprising a plurality of different perfume raw materials. Typical perfume levels in the present invention are 0.25 to 5%. Nonlimiting examples of fragrance materials include any known fragrances in the art or any otherwise effective fragrance materials. Typical fragrances are described in Arctander, “Perfume and Flavour Chemicals (Aroma Chemicals)”, Vol. I and II (1969) and Arctander, “Perfume and Flavour Materials of Natural Origin” (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314), aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Suitable fragrance materials may also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the antiperspirant compositions may comprise fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof. Fragrance materials other than those listed above may also be employed.

The antiperspirant compositions can also include residue-masking agents to reduce the appearance of white residue arising from the antiperspirant active and structurant employed in the product. These masking agents can be incorporated into either the continuous or disperse phased depending on their water solublity. Exemplary residue-masking agents include isostearyl isostearate, glycereth-7-benzoate, C12-C15 alkyl benzoate, octyldodecyl benzoate, isostearyl lactate, isostearyl palmitate, benzyl laurate, laureth 4, laureth 7, oleth 2, PEG 4, PEG 12, isopropyl myristate isopropyl palmate, butyl stearate, polyethylene glycol methyl ethers, PPG 2 ceteareth 9, PPG 2 isodeceth 12, PPG 5 butyl ether, PPG 14 butyl ether, PPG 15 butyl ether, PPG 53 butyl ether, octyldodecanol, polydecene, mineral oil, petrolatum, phenyltrimethicone, dimethicone copolyol, and mixtures thereof. One preferred concentration level of the optional residue-masking agent is from about 3% to about 10%, by weight of the composition. But other concentration levels may also be used.

Antiperspirant compositions of the present invention may employ one or more additional ingredients. Nonlimiting examples of such optional ingredients include, but are not limited to, pH buffering agents, additional malodor controlling agents, emollients, humectants, soothing agents, dyes and pigments, medicaments, baking soda and related materials, preservatives, and soothing agents such as aloe vera, allantoin, D-panthenol, pantothenic acid derivatives (e.g., those disclosed in U.S. Pat. No. 6,495,149), avocado oil and other vegetative oils, and lichen extract.

### VI. Product Clarity

Antiperspirant products made in accordance with the present invention may be opaque, translucent, or transparent. In one preferred embodiment, a 1 cm thick portion/sample of the antiperspirant product has at least 1% light transmission at 580 nm and 22° C. The following test method can be used to determine light transmission exhibited by the antiperspirant products and/or portions thereof. While still mobile, pour a sample of an antiperspirant composition into a 4.5 ml cuvette made of polymethylmethacrylate and allow to cool to a temperature of 22° C. Such a cuvette gives a 1 cm thickness of the composition. Measurement is to be carried out at 580 nm, with an identical but empty cuvette in the reference beam of a dual-beam spectrophotometer, after the sample has been held for 24 hours.

### VII. Methods for Manufacturing Antiperspirant Compositions

The description and appended claims include a listing of steps with either letter or numerical designations associated with the individual steps. It is to be understood that although they may, the methods and steps do not necessarily need to be performed in the order as shown in the figures, order of listing, or in accordance with their associated designations; for example, a step (d) may be performed before or after a step (b). Furthermore, although steps are listed individually, some steps may be performed simultaneously with other steps. Alternatively, the steps are all performed sequentially. Timing of the steps can vary. Also, there may or may not be delays between steps. And the methods described herein may include other steps than those explicitly listed and/or recited in the appended claims.

One exemplary method for making antiperspirant emulsion sticks of the present invention include the following steps: a) providing a water-immiscible liquid; b) providing a solution comprising an antiperspirant active dissolved in a polar solvent, wherein the conductivity level of the solution is less than or equal to about 75 mS/cm at 25° C., and wherein the antiperspirant active has a combined peak 4 and peak 5 area percentage of at least about 25% relative to the area sum of peaks 1 to 5; c) preparing an emulsion comprising a continuous phase including the water-immiscible liquid and a disperse phase including the solution; d) providing the emulsion with a structurant; and e) heating the emulsion to a temperature above about 80° C. The emulsion may be cooled through an active step—that is, for example, via exposure to forced air, passage through a cooled environment or the like. Otherwise the emulsion is allowed to cool simply through radiation and/or conductive heat transfer. After cooling the antiperspirant emulsion will preferably be in the form of a solid or semi-solid product that can be applied to a user's underarm.

Another exemplary method for making antiperspirant emulsion sticks of the present invention include the following steps: a) providing a water-immiscible liquid; b) providing a solution comprising an antiperspirant active dissolved in a polar solvent, wherein the conductivity level of the solution is less than or equal to about 75 mS/cm at 25° C., and wherein the antiperspirant active comprises a calcium salt, a strontium salt, or a mixture thereof; c) preparing an emulsion comprising a continuous phase including the water-immiscible liquid and a disperse phase including the solution; d) providing the emulsion with a structurant; and e) heating the emulsion to a temperature above about 80° C. Similar to the method above, the emulsion may be cooled through an active step—that is, for example, via exposure to forced air, passage through a cooled environment or the like. Otherwise the emulsion is allowed to cool simply through radiation and/or conductive heat transfer.

### VIII. Examples

The following examples further describe and demonstrate embodiments within the scope of the present invention. The examples are given solely for the purpose of illustration and are not to be construed as limitations of the present invention as many variations thereof are possible without departing from the spirit and scope of the invention.

| Ingredient | | | | | |
|---|---|---|---|---|---|
| Part I: Partial | Comparative | Comparative | | Example | Example |
| Continuous Phase | Example 1 | Example 2⁹ | ExampleA | B | C |
| Hexamethyldisiloxane¹ | 22.65 | 21.25 | 21.25 | 21.25 | 21.25 |
| DC5200² | 1.20 | 1.20 | 1.20 | 1.20 | |
| Fragrance | 1.35 | 2.25 | 2.25 | 2.25 | 2.25 |
| Shin Etsu KF 6038³ | | | | | 1.20 |
| Part II: Disperse | | | | | |
| Phase | | | | | |
| ACH (40% solution)⁴ | 40.00 | 55.0 | | | |
| IACH (34% solution)⁵ | | 2.30 | 49.00 | | |
| ZAG (30% solution)⁶ | | | | 52.30 | 52.30 |
| propylene glycol | 5.00 | | 5.00 | 5.00 | 5.00 |
| water | 12.30 | | 3.30 | | |
| Part III: Structurant | | | | | |
| Plus Remainder of | | | | | |
| Continuous Phase | | | | | |
| FinSolve TN | 6.50 | 6.00 | 6.50 | 6.00 | 6.50 |
| Ozocrite Wax | | | | | 12.00 |
| Performalene PL⁷ | 11.00 | 11.00 | 12.00 | 12.00 | |
| Aqueous Phase | 37.7 | 79.5 | 40.5 | 60.3 | 60.3 |
| Conductivity (mS/cm) | | | | | |
| Combined Peak 4 and 5 | 16 | 31.8 | 74.4 | 67.19 | 67.19 |
| area⁸ | | | | | |

¹DC 246 fluid from Dow Corning
²from Dow Corning
³from Shinetsu
⁴Standard aluminum chlorohydrate solution
⁵IACH solution stabilized with calcium
⁶IZAG solution stabilized with calcium
⁷from New Phase Technologies
⁸values are estimates for Comparative Examples 1 and 2, and actual measurements for Examples A, B and C
⁹emulsion broke when manufacturing this composition

All of the above examples can be made via the following general process, which one skilled in the art will be able to alter to incorporate available equipment. The ingredients of Part I and Part II are mixed in separate suitable containers. Part II is then added slowly to Part I under agitation to assure the making of a water-in-silicone emulsion. The emulsion is then milled with suitable mill, for example a Greeco 1 L03 from Greeco Corp, to create a homogenous emulsion. Part III is mixed and heated to 88° C. until the all solids are completely melted. The emulsion is then also heated to 88° C. and then added to the Part 3 ingredients. The final mixture is then poured into an appropriate container, and allowed to solidify and cool to ambient temperature.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A solid antiperspirant product, comprising:
(a) a continuous phase comprising a water-immiscible liquid and a structurant;
(b) a disperse phase comprising an antiperspirant active dissolved in a polar solvent,
wherein the disperse phase has a conductivity of less than or equal to about 75 mS/cm at 25° C., and
wherein the antiperspirant active has a combined peak 4 and peak 5 area percentage of at least about 25% relative to the area sum of peaks 1 to 5.

2) The solid antiperspirant product of claim 1, wherein the water-immiscible liquid comprises a volatile silicone having a flash point above about 80° C.

3) The solid antiperspirant product of claim 1, wherein the water-immiscible liquid has a flash point that is higher than the melting point of the structurant.

4) The solid antiperspirant product of claim 1, wherein the water-immiscible liquid is selected from the group consisting of cyclohexamethylsiloxane, hexyl methicone, capryl methicone and linear or branched polydimethyl siloxanes containing 4 to 6 silicone atoms.

5) The solid antiperspirant product of claim 1, wherein the structurant has a melting point that is equal to or greater than about 77° C.

6) The solid antiperspirant product of claim 1, wherein the structurant is selected from the group consisting of a polyethylene wax, an ozokerite wax, a carnuba wax, and mixtures thereof.

7) The solid antiperspirant product of claim 1, wherein the structurant comprises a fibre-forming structurant material.

8) The solid antiperspirant product of claim 1, wherein the structurant comprises a cellobiose compound.

9) The solid antiperspirant product of claim 1, wherein the disperse phase has a conductivity of less than or equal to about 60 mS/cm at 25° C.

10) The solid antiperspirant product of claim 1, wherein the antiperspirant active comprises calcium, strontium, or a combination thereof.

11) The solid antiperspirant product of claim 1, wherein the antiperspirant active comprises an aluminum-zirconium active having a combined peak 4 and peak 5 area percentage of at least about 25% relative to the area sum of peaks 1 to 5.

12) The solid antiperspirant product of claim 1, wherein the antiperspirant active comprises an aluminum-zirconium active having a combined peak 4 and peak 5 area percentage of at least about 30% relative to the area sum of peaks 1 to 5.

13) The solid antiperspirant product of claim 1, wherein the antiperspirant active comprises an aluminum only active having a combined peak 4 and 5 area percentage of at least about 25% relative to the area sum of peaks 1 to 5.

14) The solid antiperspirant product of claim 1, wherein the antiperspirant active comprises an aluminum only active having a combined peak 4 and 5 area percentage of at least about 30% relative to the area sum of peaks 1 to 5.

15) The solid antiperspirant product of claim 1, wherein the antiperspirant active has a combined peak 4 and 5 area percentage of at least about 65% relative to the area sum of peaks 1 to 5.

16) The solid antiperspirant product of claim 1, wherein a 1 cm thick portion of the solid antiperspirant product has at least 1% light transmittance at 580 nm and 22° C.

17) A solid antiperspirant product, comprising:
(a) a continuous phase comprising a water-immiscible liquid and a structurant;
(b) a disperse phase comprising an antiperspirant active dissolved in a polar solvent,
wherein the disperse phase has a conductivity of less than or equal to about 75 mS/cm at 25° C., and
wherein the antiperspirant active comprises an aluminum salt, and a calcium salt and/or strontium salt.

18) The solid antiperspirant product of claim 17, wherein the disperse phase has a conductivity of less than or equal to about 60 mS/cm at 25° C.

19) A method for making an emulsified antiperspirant product comprising a continuous phase that includes a water-immiscible liquid and a disperse phase comprising an antiperspirant active dissolved in a polar solvent, the method comprising the steps of:
(a) providing a water-immiscible liquid;
(b) providing a solution comprising an antiperspirant active dissolved in a polar solvent, wherein the conductivity level of the solution is less than or equal to about 75 mS/cm at 25° C., and wherein the antiperspirant active has a combined peak 4 and peak 5 area percentage of at least about 25% relative to the area sum of peaks 1 to 5;
(c) preparing an emulsion comprising a continuous phase including the water-immiscible liquid and a disperse phase including the solution;
(d) providing the emulsion with a structurant; and
(e) heating the emulsion to a temperature above about 80° C.

20) The method of claim 19, wherein the solution has a conductivity of less than or equal to about 60 mS/cm at 25° C.

21) The method of claim 19, wherein the antiperspirant active has a combined peak 4 and peak 5 area percentage of at least about 65% relative to the area sum of peaks 1 to 5.

22) A method for making an emulsified antiperspirant product comprising a continuous phase that includes a water-immiscible liquid and a disperse phase comprising an antiperspirant active dissolved in a polar solvent, the method comprising the steps of:
(a) providing a water-immiscible liquid;
(b) providing a solution comprising an antiperspirant active dissolved in a polar solvent, wherein the conductivity level of the solution is less than or equal to about 75 mS/cm at 25° C., and wherein the antiperspirant active comprises a calcium salt, a strontium salt, or a mixture thereof;
(c) preparing an emulsion comprising a continuous phase including the water-immiscible liquid and a disperse phase including the solution;
(d) providing the emulsion with a structurant; and
(e) heating the emulsion to a temperature above about 80° C.

23) The method of claim 22, wherein the solution has a conductivity of less than or equal to about 60 mS/cm at 25° C.
